(19) United States
(12) Patent Application Publication
(10) Pub. No.: US 2005/0013853 A1
(43) Pub. Date: Jan. 20, 2005

(54) Anti-proliferative drugs

(76) Inventors: Irit Gil-Ad, Herzelia (IL); Abraham Weizman, Tel Aviv (IL)

Correspondence Address: Gary M. Nath;NATH & ASSOCIATES PLLC, 6th Floor, 1030 15th Street, N.W., Washington, DC, 20005, US

(21) Appl. No.: 10/774,694

(22) Filed: Feb. 10, 2004

Related U.S. Application Data
(63) Continuation-in-part of application No. 10/432,875, filed on Sep. 16, 2003.

(30) Foreign Application Priority Data
Nov. 29, 2000 (IL) 139975

(51) Int. Cl. A61K 9/70

(52) U.S. Cl. 424/449; 514/649

(57) ABSTRACT

The present invention concerns topical compositions comprising as an active ingredient at least one cyclic psychotropic agent, preferably an anti-depressant or an anti-psychotic drug. The invention further concerns a method for the treatment of dermatological diseases or disorders, such as hyper-proliferative or inflammatory skin diseases, by topical application of a cyclic psychotropic agent

### FIELD OF THE INVENTION

The present invention is generally in the field of pharmaceutical compositions and in the field of methods for the treatment of a disease or disorder. In particular the present invention concerns topical compositions for the treatment of skin diseases or disorders.

### BACKGROUND OF THE INVENTION

Psychotropics are drugs used mostly for therapy of psychiatric disorders such as schizophrenia and mood disorders. Some psychotropic drugs exert their activity by blocking D2 dopaminergic receptors and inactivating dopamine neurotransmission in forebrain. Other drugs may act through interaction with D1-dopaminergic receptors, 5-HT2 serotonergic receptors and α-adrenergic receptors. Selective sertotonin reuptake inhibitors (SSRIs), such as paroxetine, sertraline, and fluoxetine are the most commonly prescribed antidepressants and are considered as highly effective and relatively safe.

The therapeutic effect of some psychotropic drugs, administered systemically, on proliferative disorders has already been described. For example, Silver et al., [Society of Biological Psychiatry, 35:824-826, (1999)] showed the inhibitory effect of anti-psychotic drugs, such as haloperidol, flupenthixoland fluphenazine on human neuroblastoma cell lines. Other studies showed that phenothiazines administered systemically have anti-proliferative effects on some tumor cells such as leukemic cells, melanoma, glioma and leukemia [Nordenberg et al., Biochemical Pharmacology, 58:1229-1239, (1999)].

With regard to the effect of antidepressants on hyper-proliferation, conflicting reports exist. Clomipramine, imipramine and citalopram were found to induce apoptosis in myeloid leukemia HL-60 cells [Xia et al. J. Biochem Mol Toxico.l 13:338-47 (1999]. In addition, the monocyclic serotonin reuptake inhibitors fluoxetine and zimelidine were shown to inhibit proliferation of prostate carcinoma cells [Abdul M et al. J. Urol. 154:247-50 (1995)]. However, other studies showed an opposite effect that in vivo administration of fluoxetine and amitryptiline to mice increased the development of fibrosarcoma, melanoma and breast tumors [Brandes L J, et al. Cancer Res. 52:3796-00 (1992)]. In addition, it was shown that antidepressant medications (some tricyclic antidepressants (TCAs) and paroxetine) are either associated with elevation of risk in breast cancer [Cotterchio M. et al., Am J Epidemiol, 151:951-7 (2000)], or are non-effective in decrease of cancer [Wang P. S. J. Clin. Epidemiol. 54:728-34, (2001)].

The systemic use of psychotropic drugs in treating patients with skin disorders having clear discernible psychiatric symptoms has also been described. Examples include systemic treatment of psoriasis associated with major depression by the administration mainly of TCAs and SSRIs [Gupta M. A. et al. J. Am. Dermatol. 14(4):633-645 (1986); and the treatment vitiligo resulting from social anxiety and delusions of parasitosis Tennyson H and Levine N. Dermatol. Clin. 19(1):179-197 (2001)] by administration of TCAs, SSRIs, naltrexone, pimozide, and gabapentin. In connection with these diseases as well, conflicting data exists and some systemically administered psychotropic drugs were found to induce skin diseases such as the inducing effect of systemically administered fluoxetine on psoriasis [Hemlock C. et al. Ann. Pharmacother. 26(2):211-212 (1992)].

Several publications also described the systemic use of psychotropic agents in treating patients with alopecia areata and psychiatric comorbidity. Other studies indicate the beneficial effects of systemic administration of imipramine, a tricyclic antidepressant, or citalopram, an antidepressant from the SSRIs group, on regrowth of hair in alopecia areata patients (Perini G I et. al. Psychother Psychosom. 96:33-34 (1994); Doblado S R et. al. Int. J. Dermatol. 38:798-799 (1998)).

However, as in other cases there exists conflicting evidence, for example, systemic administration of seroxat caused alopecia (Umansky L et. al. Harefuah 138(7):547-549 (2000)).

In all the above cases, the effect (either beneficial or undesired) of psychotropic drugs on the skin was exerted by psychotropic drugs administered systemically.

In International Patent Publication No. WO01/66101, the systemic effect of some monocyclic antidepressant drugs commonly linked by their norepinephrine reuptake inhibiting activity, on treatment of dermatological disorders is described. Specifically, this publication describes the systemic administration of several norepinephrine reuptake inhibitors on psoriasis (e.g. oral). This publication interacts a single example of a topical composition comprising tomoxetine (today termed “atomoxetine”) (without physiological evidence) which presumably is also intended for trasdermal administration.

Finally, International Patent Publication No. WO01/87236 describes cream and spray formulations include antihistaminic agent such as hydroxyzine together with ketotifene or the tricyclic antidepressant doxepine for treating of Pruritus without eliciting side effects typically associated with medicaments for symptomatic skin itching (e.g. weight gain, sedation).

### SUMMARY OF THE INVENTION

The present invention is based on the surprising finding that a large number of cyclic psychotropic agents, more specifically cyclic antipsychotic and anti-depressants, having a wide scope of chemical structures, all have a beneficial effect in a large number of dermatological diseases and disorders, when applied topically onto the skin.

The surprising finding, that systemically administered cyclic psychotropic agents are effective topically, paves the way to the establishment of an effective treatment of a large number of dermatological diseases and disorders by topical application of said cyclic psychotropic agents.

Topical administration of such agents, contrary to their systemic administration ,has the desired result that the therapeutic effect is mostly limited to the diseased skin areas, and systemic effect, which is to be avoided in order to eliminate undesired effects on the CNS, is thus minimized.

This finding is extremely surprising, as many of the cyclic psychotropic agents, such as serotonin re-uptake inhibitors (SSRI) noradrenergic re-uptake inhibitors (NARI), mixed serotonin and noradrenalin re-uptake inhibitors (SNRI), tricyclic antidepressants (TCA) and atypical antidepressants, have no known receptor targets in skin cells, so that their mode of action and their interaction with effector molecules and signal transduction pathways are currently unknown.

Without wishing to be bound by theory, it is assumed that these cyclic psychotropic agents exert their therapeutic effect on the skin, through one of the following mechanisms:
(1) where the dermatological disease, disorder or pathological condition is mainly due to hyperproliferation (such as in cases of psoriasis, hypercarothosis, skin cancer such as melanoma, basal cell carcinoma and additional proliferative skin disorders) the mechanism is mainly believed to be due to enhanced apoptosis, and/or modulation of cell cycle of the hyproliferative cells so as to increase the proportion of cells in the G0/G1 arrest stage;
(2) where the skin disease, disorder or pathological condition is mainly manifested by, or associated with, inflammation, the therapeutic activity of the drug is believed to be caused either by anti-proliferative effect on lymphocytes, (due to increased apoptosis or increased G0/G1 arrest), or due to the inhibitory effect on cytokine production in lymphocytes, keratinocytes or any other skin cells as will be explained here in below.

Thus, by one aspect, the present invention concerns a topical pharmaceutical composition, comprising a topically acceptable carrier and as an active ingredient, a cyclic psychotropic agent, said cyclic psychotropic agent being other than doxepine and atomoxetine.

The present invention also concerns a method for the treatment of a dermatological disease, disorder, or pathological condition the method comprising, topically administering to a patient in need of dermatological treatment, a therapeutically effective amount of a psychotropic cyclic agent, said cyclic psychotropic agent being other than doxepine and atomoxetine.

More specifically the present invention concerns a topical pharmaceutical composition for administration to mucosal tissue (such as to the epithelial cells of the vagina, rectum, nose, mouth, ear, inner lid), comprising a topically acceptable carrier and as an active ingredient, a cyclic psychotropic agent, said cyclic psychotropic agent being other than norfluxetine.

More specifically, the present invention concerns a method for the treatment of hyper-proliferative dermatological diseases, disorders or pathological conditions, comprising topically administering to a subject, in need of such treatment, a therapeutically effective amount of a cyclic psychotropic agent,
wherein where the hyper-proliferative skin disorder is psoriasis, the cyclic psychotropic agent is not atomoxetine

The present invention further concerns a method for the treatment of an inflammatory dermatological disease, disorder or pathological condition comprising topically administering to a subject, in need of such treatment, a therapeutically effective amount of a cyclic psychotropic agent,
wherein where the inflammatory skin disorder, disease or pathological condition is manifested by pruritus, the cyclic psychotropic agent is not doxepine.

Preferably the present invention concerns a method for the treatment of an inflammatory dermatological disease, disorder or pathological condition comprising topically administering to a subject, in need of such treatment, a therapeutically effective amount of a cyclic psychotropic agent,
wherein where the inflammatory skin disorder, disease or pathological condition is manifested by pruritus or by atopic dermatitis, the cyclic psychotropic agent is not doxepine

The surprising finding that cyclic psychotropic agents have a dermatological effect when applied topically, paves the way to the use of cyclic psychotropic agents likely as candidates, in the search for additional topically effective dermatological drugs.

It should be obvious to any person versed in the art, that some cyclic psychotropic agents are more effective, as topically applied dermatological agents than others. It should further be evident to any person versed in the art that some cyclic psychotropic agents are more effective when topically applied for the treatment of one specific dermatological condition, as compared to their efficacy in the treatment of another specific dermatological condition.

A man versed in the art, once aware of the teaching of the present invention, will have no problem in finding which cyclic psychotropic agent is more effective than the other for the treatment of a particular disease, and which cyclic psychotropic agent is more effective for a specific disease as compared to its efficacy in the treatment of another disease.

The present invention is further based on the finding that antipsychotic drugs administered topically can sensitize the skin cancer cells to subsequent treatment (also subsequent systemic treatment) by chemotoxic drugs.

By another aspect, the present invention concerns use of topically applied cyclic anti-psychotic drugs, other than fluoxetine,, for the for sensitizing multidrug resistance cancer skin cells cells, such as melanoma cells to adriamycin (doxorubicin) and other cytotoxic agents (which may be administered systemically).

U.S. Pat. No. 6,630,454 discloses the use of fluxetine for the chemosensitization of MDR cells but is silent as regards other cyclic osychotrophic agents.

More specifically the cyclic anti-psychotic drugs are phenothiazines In particular, the phenothiazines, in accordance with the invention, are of the following: thioridazine, chloropromazine, trifluoperazine, flupentixol, fluphenazine and perphenazine.

The drugs, , can be administered topically both during the active chemotherapy stage, optionally together with other known anti-tumor agents having an anti-proliferative activity, as well as for secondary prevention purposes (chronic intake) during remission states. The drugs may be especially useful for treatment of skin cancers which are resistant to doxorubicin and other chemotoxic agents, since they are capable of effecting even those skin cancers which are drug resistant.

Therefore the present invention further concerns a combination of topicaly cyclic antipsychotic drug drugs and a cytotoxic agent for the treatment of multi-drug resistant cancer. In particular the drugs are tricyclic antipsychotic drugs or phenothiazine, for example as indicated above and the cytotoxic drug is doxorubicin.

Thus, by yet another aspect, the present invention concerns a method for identifying and screening an active agent for the treatment of a dermatological disease, disorder or pathological condition by topical application and screening of suitable candidates, the method comprising:
(a) providing one cyclic psychotropic drug as a candidate active agent;
(b) applying the cyclic psychotropic drug to a biological model system for said dermatological disease, disorder or pathological condition;
(c) monitoring the change in at least one physiological parameter, said change being indicative of a beneficial therapeutic effect in said biological model system;
wherein a significant change in said at least one physiological parameter as compared to control indicates that the candidate cyclic psychotropic agent is active for the treatment of said dermatological disease, disorder or pathological condition.

The biological model system may be non-cellular such as molecular components of a signal transduction pathway ,known to be involved in said dermatological disease, disorder or pathological condition; an in vitro model system relevant to said disease; an in vivo model relevant to the disease; or clinical trials in humans.

The monitoring should be of at least one physiological parameter known to be relevant to the specific disease wherein the direction of the change of the parameter (increase/decrease) is known to be correlated with a beneficial therapeutic effect.

Where the diseases are proliferative, the model may be an in vitro system of keratinocytes (for example HaCat cells) and the physiological parameter may be viability of cells measured by cell count, level of apoptosis, (DNA fragmentation, caspase 3 activation, etc.) % of cells in each cell cycle (monitoring by FACS) etc.

Where the disease is a malignant proliferative disease the model may be an in vitro model such as a cell lines of said specific skin cancer (for example B16 melanoma cells) and the viability of cells measured by cell count, level of apoptosis, (monitored by DNA fragmentation, caspase 3 activation, etc.) % of cells in each cycle. (monitored by FACS) etc.

Alternatively, the model may be in vivo for skin cancer, such as chemical-induced skin cancer (e.g. porobol esthers such as TPA , Dinitrobenzoic acid (DNBA)) or transgenic animals (e.g. TGAC mouse model), and in vivo models for psoriasis such as essential fatty acid-deficient hairless rat, transgenic mice, mutant strains of mice and severe combined immunodeficiency (SCID) mouse-human skin chimeras (xenotransplantation model)).

Where the disease is an inflammatory disease, the model may be an in vitro model such as Con-A induced lymphocyte or splendocytes proliferation and the physiological parameter measured is the number of cells.

Another physiological parameter in the same model system is the type and amount of the cytokines (IL-2, INF-γ, TNFα, etc.) which are secreted from lymphocytes, kemocytes, fibroblasts, mast cells or splenocytes.

Where the disease is an inflammatory an example of an in vivo model for topical applications is chemically-induced skin dermatitis and severe combined immunodeficiency (SCID) mouse-human skin chimeras for the study of cutaneous inflammation.

### GENERAL DESCRIPTION OF THE INVENTION

Glossary:

In the following, the terms mentioned hereinafter, will be used with the following meaning:

“Dermatological disease, disorder or pathological condition” refers to any disease that impairs the normal function of the skin. This term also refers to a disease impairing the epithelial cells of the mucosal tissue (a local effect in the mucosal tissue without any desired trans-mucosal delivery). The local mucosal effect may be on the epithelial cells of the nose, mouth, ear, vagina, rectum, inner lid etc. This term refers to any disease which affects any of the cell-types present in the epidermis or dermis (of the skin or the mucosal tissue), and more specifically a disease which impairs the function of epithelial cells, keratinocytes, lymphocytes, melanocytes, fibroblasts, mast cells, langerhans cells and other cell types of epidermis and dermis such as hair follicles, sebaceous glands, and sweat glands. The disease, disorder or pathological condition may be the main manifestation of the disease, such is in the case of psoriasis, or may be merely the dermatological manifestation of another disease which is not necessarily dermatologic in origin, for example, a dermatological manifestation of rheumatoid arthritis (for example leg ulceration).

“Hyper-proliferation”—the dermatological disease, disorder or pathological condition may be a disease associated with hyper-proliferation of the skin cells. Without being limited to the following examples, hyper-proliferative skin disorders, diseases or pathological conditions include psoriasis, epidermal hyperplasia, hyperkeratosis, acanthosis, papilloma (such as women's mucosal tissue of the vagina, as well as genital diseases, cervix carcinoma), scleroderma, actinic keratoses, and skin cancer. The skin cancer may include, without being limited thereto, basal cell carcinoma, melanoma, squamous cell carcinoma, cutaneous T-cell lymphoma and Kaposi's sarcoma.

“Inflammatory”—Alternatively, the skin disorder, disease or pathological condition may be associated with inflammation, i.e. may be the dermatological manifestation of an inflammatory, and/or autoimmune disease, or may be an inflammatory skin disorder which sole, or main manifestation is in the skin (or epidermal cells of the mucosal tissue). Specific, non-limiting examples of autoimmune skin disorders or pathlogical conditions include scleroderma, vitiligo, alopecia areata, psoriatic arthritis, lichen planus, lichen sclerosus, cicatrical pemphigoid discoid lupus, lupus erythematosus, leg ulceration in rheumatoid arthritis, atopic dermatitis and pyoderma gangrenosum. The inflammation disorder may be non-autoimmune diseases such as rosacea, urticaria, contact dermatitis and seborrheic dermatitis.

The term “treatment” as used herein refers to the topical application of a therapeutic amount of the active agent according to the invention which is effective in one of the following: ameliorating undesired symptoms associated with the dermatological disease, disorder, or pathological conditions ;effective in preventing the manifestation of such symptoms before they occur (for example to prevent remission of acute phase of autoimmune-associated dermatological conditions); effective in slowing down the progression of the disease or disorder (for example slowing progress of skin cancers); effective in slowing down the deterioration of the disease (for example effective in restricting the spreading of psoriasis to healthy region); effective to prolong the time period onset of remission period (especially in autoimmune diseases such as lupus); effective in slowing down the irreversible damage caused in the progressive chronic stage of the disorder; effective to delay the onset of said progressive stage (for example delaying the onset of melanoma stage IV); effective to lessen the severity or cure the disease or disorder; effective to improve survival rates of individuals infected with the disease, or effective to prevent the disease form occurring altogether (for example in an individual generally prone to the disease) or a combination of two or more of the above.

The term “topical” (topical administration/topical treatrnent/topical agent) according to the invention concerns the application of an active agent on the skin or on the epithelia cells of the mucosal tissue (mouth, nose, ear, vagina, rectum, inner lid, nose) for effecting physiological parameters (proliferation of cells, production of agents, such as cytokines) of cells present in the skin/epithelial layer of the mucosal tissue (both epidermis and in the dermis). This application is intended to affect the skin or said epithelial cells of the mucosal tissue and no transdermal or tranmucosal effect is desired.

The term “topically acceptable carrier” refers to any vehicle, adjuvant, excipient, diluent, which is known in the field of pharmacology for application onto the skin (or the epithelial layer of the mucosal tissue ) and is approved for dermal/mucosal administration. The choice of carrier will be determined by the particular active agent, for example, its dissolution in that specific carrier (hydrophilic/hydrophobic), as well as by other criteria such as the size and the nature of the area to which it should be applied (for example in the scalp shampoos may be used while for small area a salve is more applicable, etc.). The topical composition may be applied to the target skin cells according to any conventional mode of topical administration. This includes, without being limited thereto, ointment, cream, gel, solution, suspension, lotion, shampoo, foam, lyposomic formulation, paste, emulsion, salve. Additional formulations may be used where the pharmaceutical composition is to be applied locally to the epithelail cells of the mucosal tissue for producing a local effect such as, suppositories, vaginal tablets, ocular salves or drops, otic drops, nasal spray, nasal drops.

The term “therapeutically effective amount” refers to an amount which causes the desired therapeutic effect (where it may have an anti-proliferative effect, anti-inflammatory effect or another desired effect), when topically/mucosal applied to the skin.

Preferably the amount of the active ingredient is 0.01-1% w/w of the total weight of the formulation preferably 0.05 to 1.0% most preferably 0.07 to 0.5%

It should be noted that in accordance with the invention, the main effect of the topical administration should be on skin cells (or the epithelial cells of the mucosal tissue) , and the systemic effect, for example caused by transdermal/trans-mucosal delivery of the compound beyond the dermis, is reduced in order to minimize undesired effects on the CNS. It should be appreciated that the aim of the formulator preparing the composition, and of the physician prescribing the topical composition comprising cyclic psychotropic drug of the invention, for the treatment of dermatological conditions, is to minimize as much as possible transdermal delivery of the drug into the circulation, and in particular to minimize, as much as possible, the delivery of the psychotropic agent to the central nervous system.

Minimizing the systemic effect caused by topical applications can be achieved by bringing into consideration the following criteria according to Pick's Law:

$$J = \frac{DKCs}{AH} .$$

J=Transfer through membrane
(a) Nature of the drug (D): the specific nature of the drug chosen should be considered, in particular with reference to the size of the cyclic psychotropic drug, its hydroprobicity, its charge, so as to minimize transdermal delivery and hence systemic effect;
(b) Nature of the carrier (K): the nature carrier should be chosen so that the partition coefficient of the drug between the carrier and the skin is such that on the one hand the active ingredient may affect the skin, but on the other hand the drug will not be delivered transdermally into the circulation;
(c) Concentration of the drug (Cs): the concentration of the drug of the carrier should be chosen with respect to the other parameters, so that the concentration gradient, and hence the flux of the active ingredient from the composition into the skin, is such as to minimize transdermal delivery;
(d) Size of area treated (A): The size area of the skin to be treated should be considered in connection with all the above criteria—i.e. the larger the area to be treated, the more important it is to minimize transdermal delivery and hence systemic effect. Another important consideration is the area of the body where the drug is applied (H), as it is known that in some areas, absorption into deeper transdermal layers is more pronounced as compared to others;
(e) Duration of treatment: It is clear that there is a cumulative effect of the drug and a drug that is to be administered for longer periods of time for treatment of a chronic disease such as psoriasis, should comply with more severe criteria of (a)-(d) as compared with a drug to be administered for treatment of an acute condition such as inflammatory condition.

As some of the criteria of (a)-(e) have opposite effects, when deciding eventually on the best drugs, carrier, concentration, duration of treatment, all the above criteria should be brought into consideration.

### CLASSIFICATION OF AGENTS

The term “cyclic psychotropic agent” as used herein refers to a chemical compound having at least one aromatic ring, or two or three conjugates rings, which is commonly used as a CNS active agent that exerts its effect on the mind or affects the mental state. More specifically such psychotropic agents are sued as antipsychotic and antidepressants.

The cyclic psychotropic agents may be classified either by their physiological mode of action or by their general chemical structure and some agents are classified by both modes of actions.

In the following where the name of a specific psychotropic drug is given it should be understood as referring not only to the formula of the agent as given for example in chemical abstracts or medicinal manuscripts (e.g. in Psychotropic 2000/2001 Lundbck Ed.) but also to small modifications in the formula such as those which increase stability; increase permeability of the compound to cells or decrease permeability to the blood brain barrier (BBB) and nevertheless, maintain the biological activity of the agent against diseased cells. The modifications in the formula may also include physiologically acceptable salts of the compound as known in the art of pharmacy, solvents, clathrates such as hydrates, enantiomers and stero-isomers, crystalline, amorphous and polymorphous configurations of the drug and metabolites of the active agents.

A: Antidepressants

Classification by Mode of Action:

The Antidepressants may be active by one of several physiological mechanisms such as: selective serotonin re-uptake inhibitor (SSRIs); selective noradrenaline re-uptake inhibitors (NRIs), serotonin and noradrenergic re-uptake inhibition (SNRI).

Examples of SSRIs include: antidepressant drugs, such as, without being limited thereto, fluoxetine, paroxetine, sertraline.

Examples of NRIs include: atomoxetine and reboxetine.

Examples of SNRIs include: venlafaxine, duloxetine and milnacipran.

Classification by General Chemical Formula:

Examples of tricyclic antidepressants (TCAS) include: imipramine, clomipramine, amitriptyline and doxepinee.

Examples of bicyclic antidepressants include: paroxetine, sertraline and citalopram

Examples of an monocyclic antidepressant include: phenylpropylamine derivatives, preferably phenoxy-3-propylamine derivatives, such as atomoxetine, nisoxetine, fluoxetine ,norfluoxetine, reboxetine or venlafaxine.

Examples of a atypical antidepressant include, mianserin, bupropion, mirtazaoin, trazodone.

Preferably, antidepressants employed according to the invention include paroxetine, fluoxetine, norfluoxetine, sertraline and clomipramine.

B. Anti-psychotic Drugs (Neuroleptic Drugs):

The agent may also be a tricyclic anti-psychotic drug or atypical anti-psychotic drug.

Example of tricyclic antipsychotic drugs include: without being limited thereto, phenothiazine or thioxanthene class of compounds preferably dibenzodia zepine derivative, or thio phenothiazine derivative. Specific examples are thioridazine, perphenazine trifluoperazine or fluphenazine. Specific examples of antipsychotics which are not of the phenothiazine class of agents include flupenthixol.

Example of atypical antipsychotic include: clozapine and clotiapine.

Preferably the anti-psychotic drug is thioridazine, trifluoperazine, flupentixol and clozapine

The active agent according to the invention is dosed in accordance with good medical practice, taking into account the clinical condition of the individual patient, the site and method of administration, scheduling of administration, patient age, sex, body weight and other factors known to medical practitioners.

The invention will now be described by way of examples with reference to the accompanying Figures. While the foregoing description describes in detail only a few specific embodiments of the invention, it will be understood by those skilled in the art that the invention is not limited thereto and that other psychotropic agents may be applied to other types of proliferative diseases, without departing from the scope of the invention as defined by the appended claims.

### BRIEF DESCRIPTION OF THE DRAWINGS

In order to understand the invention and to see how it may be carried out in practice, a preferred embodiment will now be described, by way of non-limiting example only, with reference to the accompanying drawings, in which:

FIG. 1A-1B are graphs exhibiting the effect of some psychotropic agents on viability of keratinocytes, including HaCat cells (FIG. 1A) and HaCat A5 cells (FIG. 1B);

FIG. 2A-2B are bar graphs exhibiting the effect of some psychotropic agents on viability of HaCat A5 cells (FIG. 2A) or HaCat II4 cells (FIG. 2B);

FIGS. 3A-3B are graphs exhibiting the effect of thioridazine (“Thio”), doxorubicin (“Dox”) or 5-fluorouracil (“5-FU”) on viability of keratinocytes including HaCat cells (FIG. 3A) and HaCat A5 cells (FIG. 3B);

FIG. 4 is a bar graph exhibiting the effect of two different concentrations of thioridazine (“Thio 25” refers to a concentration of 25 μM and “Thio 50” refers to 50 μM) on DNA fragmentation (% apoptosis) in HaCat cells or HaCat A5 cells, as compared to non-treated keratinocytes (“Control HaCat” or “Control HaCatA5”);

FIGS. 5A-5D are fluorescent microscope images exhibiting the effect of dexamethasone (FIG. 5A), clomipramine (FIG. 5B) and paroxetine (FIG. 5C) on cell nuclei of HaCat cells stained with propidium iodide (PI)+Hoechst, in comparison with a control, Saline-treated cells (FIG. 5A);

FIGS. 6A-6B are graphs exhibiting the effect, at two different concentrations of paroxetine (FIG. 6A) or sertraline(FIG. 6B) on caspase 3 activation in human HaCaT cells, in the presence or absence of an inhibitor (“inhib”) in comparison with a control, Saline-treated cells (“Control”);

FIG. 7 is a graph exhibiting the effect of clozapine on viability of wild type and MDR B16 melanoma cells;

FIGS. 8A-8B are graphs exhibiting the effect of some antidepressants on the viability of wild type (FIG. 8A) and MDR B16 melanoma (FIG. 8B) cells;

FIGS. 9A-9C are bar graphs exhibiting the effect of some psychotropic agents on doxorubicin-induced toxicity in wild type and MDR B16 melanoma cells, including: the effect of clomipramine on the viability of wild-type B16 melanoma cells (FIG. 9A); on MDR B16 melanoma cell (FIG. 9B); and the effect of paroxetine on wild type B16 melanoma cells (FIG. 9C);

FIG. 10. is a graph showing the effect of fluoxetine as compared to its metabolite norfluoxetine on the viability (% of control)of keratinocytes (HaCat) cells.

FIG. 11. is a graph showing the effects of paraxetine, dexamethasone, trazodon and sertraline on the viability of HaCat cells

FIG. 12 is a bar graph exhibiting the effect of paroxetine (at concentrations of 2.5, 5 and 10 μM, 0 serving as control) PHA induced human lymphocyte proliferation, the lymphocyte being obtained from three different subjects

FIG. 13 is a bar graph exhibiting the effect of some antidepressant drugs (paroxetine and sertraline), in comparison with dexamethason, on Con-A-induced mouse splenocyte proliferation

FIG. 14 is a bar graph exhibiting the effect of some antidepressant drugs, including paroxetine and clomipramine in comparison with dexamethasone on mouse splenocytes proliferation and cytokines (EL-2 or INF-γ) release

FIG. 15 is a graph showing the effect of paroxetine, sertraline thioridazine and dexamethasone (2.5-20 μM) on Con-A-induced TNF-α secretion from mouse splenocyte 48 hr after exposure to drugs

FIGS. 16A and 16B are bar graphs showing, respectively, the effect of paroxetine as compared to dexamethasone (2.5-25 μM) on Con-A-induced splenocyte proliferation (Alamar blue method), and splenocyte-induced secretion of TNF-α 48 hr after exposure to drugs.

### SPECIFIC EXAMPLES

### Example 1

### In Vitro Studies

### Example 1A

### Effect on Cell Viability in Keratinocytes

Three hunan immortal keratinocytes cell-lines (Bachmeir & Nerlich, Bachmeier B E, Nerlich A G. Int J Oncol 20(3):495-9, March 2002) were employed: HaCat (spontaneously immortalize, non tumorigenic human skin keratyniocyte line) HaCat A5 (benign, tumorigenic), and HaCat II-4RT (Malignant Tumorigenic).

These cells were maintained as described by Bachmeier B E et al. [Bachmeier B E et al. Biol. Chem 381(5-6):509-516 (2000)]. In general, 10,000 cells/well were treated with drugs from different categories of psychotropic drugs. The exemplified drugs include (the category indicated in brackets): thioridazine and perphenazine (phenothiazines); clozapine (tricyclic anti-psychotic); clomipramine, imipramine and doxepine (tricyclic antidepressants); paroxetine and sertraline (bicyclic antidepressants); fluoxetine (monocyclic antidepressant).

Drugs were provided at concentrations within the range of 5-100 μM and cell viability was measured 24 hr post-administration by Neutral Red Staining.

The effect of the drugs was compared at equimolar concentrations to that of two commonly used anticancer agents (Doxorubicin and 5-fluorouracil (5-FU)).

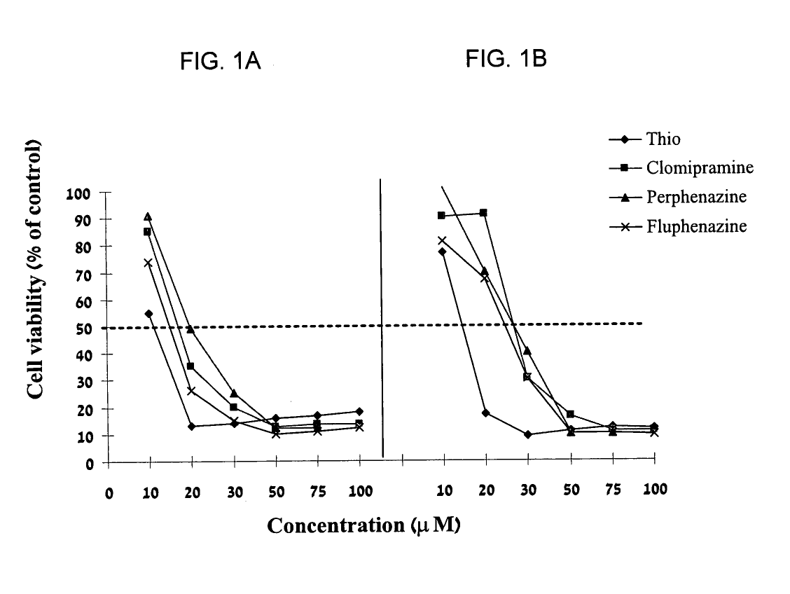

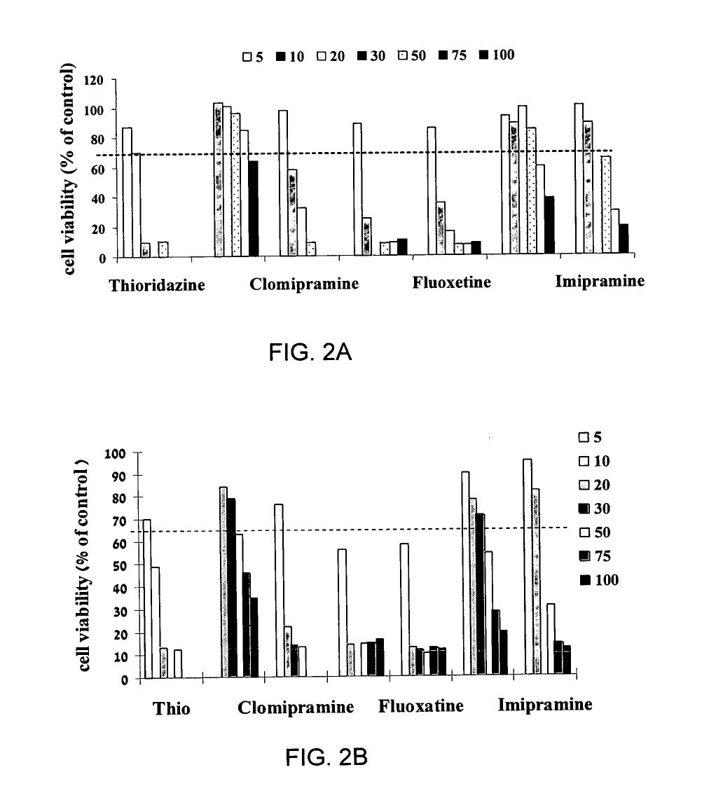

The different drugs were shown to induce a marked dose dependent inhibitory activity on viability of HaCat, HaCat A5 and HaCat II4 cell lines (FIG. 1A and FIG. 1B, and FIG. 2A and FIG. 2B respectively). As presented in these figures thioridazine (a phenothiazine),clozapine (a tricyclic antipsychotic), clomipramine and imipramine (tricyclic antidepressants) fluoxetine, (a monocyclic antidepressant) and paroxetine (a bicyclic antidepressant) were shown to be effective in reducing viability of HaCat, HaCat II4 and HaCat A5 cells The sensitivity (IC50) of the malignant tumorigenic cell line, HaCat II-4RT, to the different drugs was determined. Briefly, HaCat II4RT were shown to be more sensitive as compared to the non malignant (A5) cells or of the non tumorigenic HaCat cells (showing a sensitivity similar to that of the A5 cell line. The IC50 of the different drugs with the three keratinocyte cell lines is summarized in Table 1 (which shows the mean results of several experiments) and FIG. 2A and FIG. 2B (which show the results of a single experiment)

TABLE 1
Mean IC50 (μM) in keratinocytes

| Drug | Gategory | HaCat | HaCat A5 | HaCatII4RT |
|---|---|---|---|---|
| Thioridazine | Phenothiazines | 11.5 | 14 | 10 |
| Perphenazine | Phenothiazines | 24.0 | 26.0 | NT¹ |
| Fluphenazine | Phenothiazines | 19.0 | 22.0 | NT¹ |
| Clomipramine | TCA² | 20.0 | 22.0 | NT¹ |
| Imipramine | TCA² | NT¹ | 62.0 | 48.0 |
| Doxepinee | TCA² | NT¹ | 90.0 | 56.0 |
| Paroxetine | SSRIs BCA³ | 17.8 | 21.0 | 12.0 |
| Sertraline | SSRIs BCA³ | 24.7 | NT¹ | NT¹ |
| Citalopram | SSRIs BCA³ | >100 | NT¹ | NT¹ |
| Fluoxetine | SSRIs⁵ MCA⁴ | 19.0 | 20.0 | 13.0 |
| Venlafaxine | SNRIs⁶ MCA⁴ | >100 | NT¹ | NT¹ |
| Reboxetine | NARIs⁷ MCA⁴ | >100 | NT¹ | NT¹ |
| Clozapine | TCNA⁷ | >100 | >100 | 73.0 |
| Clotiapine | TCNA⁷ | >100 | >100 | NT¹ |

¹not tested;
²tricyclic antidepressant;
³bicyclic antidepressant;
⁴monocyclic antidepressant;
⁵Selective sertotonine reuptake inhibitor;
⁶Serotonine-Noradrenaline Reuptake Inhibitor;
⁷tricyclic neuroleptic and antipsychotic.

The IC50 values obtained for listed drugs range between 10 μM and 100 μM, with those in the range of 10-30 μM being considered the preferable.

Responsiveness in the HaCat and HaCat A5 cells to the chemotoxic agents doxorubicin and 5-FU was tested in the presence of thioridazine (FIG. 3A and FIG. 3B, respectively). Both cell-lines responded to thioridazine with a similar pattern of sensitivity, but were resistant to 5-FU. The non tumorigenic HaCat cells responded to doxorubicin with an equimolar IC50 as for thioridazine.

### Example 1B

### Effect on DNA Fragmentation in Keratinycytes

The effect of psychotropic drugs on DNA fragmentation (apoptosis) was determined by flow cytometric analysis of propidium iodide (PI)-stained cells according to the method of Vindelov et al [Vindelov, L. L., et al. Cytometry. 5:323-327 (1983)], using a fluorescence activated cell sorter (FACScan, Becton and Dickenson, Calif.). The study was conducted with HaCat and HaCat A5 cell lines (500,000 and 1,000,000 cells each sample, respectively) treated with thioridazine (25 or 50 μM). Cells provided with saline served as the control group.

HaCat cells exhibited basal fragmentation of 29% (control), however, upon treatment with thioridazine the rate of fragmentation increased to a level of 82.8% (with 25 μM) and 89.3% (with 50 μM). HaCatA5 cells exhibited basal fragmentation of 10.23% (control) and following exposure to thioridazine, fragmentation increased to 74.5% (with 25 μM) and 76.6% (with 50 μM).

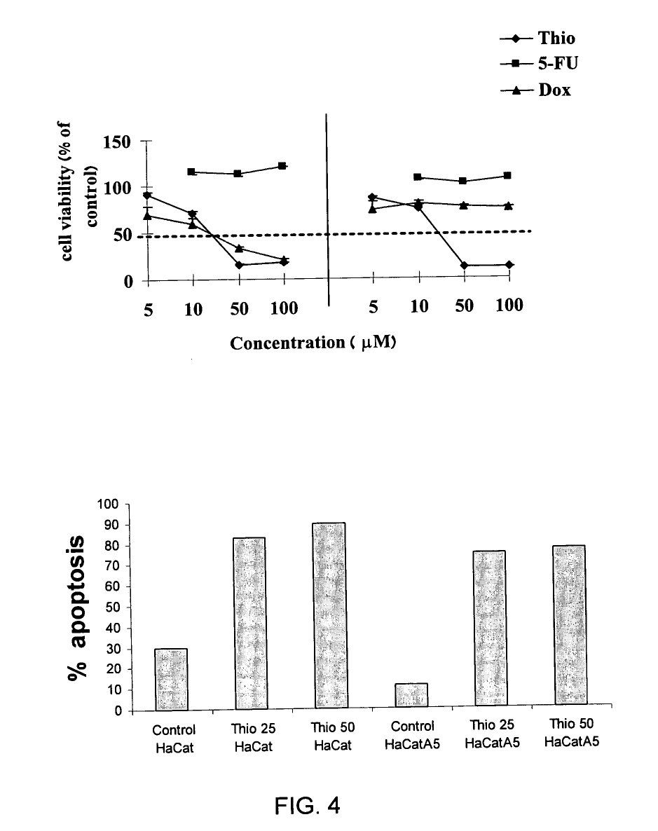

FIG. 4 presents the percentage of apoptosis (DNA fragmentation) with the HaCat and HaCat A5 under the different conditions. These results suggest that the inhibitory effect of thioridazine on the viability of proliferative skin cells is mediated by augmenting DNA fragmentation, which is a hallmark of the apoptotic mechanism.

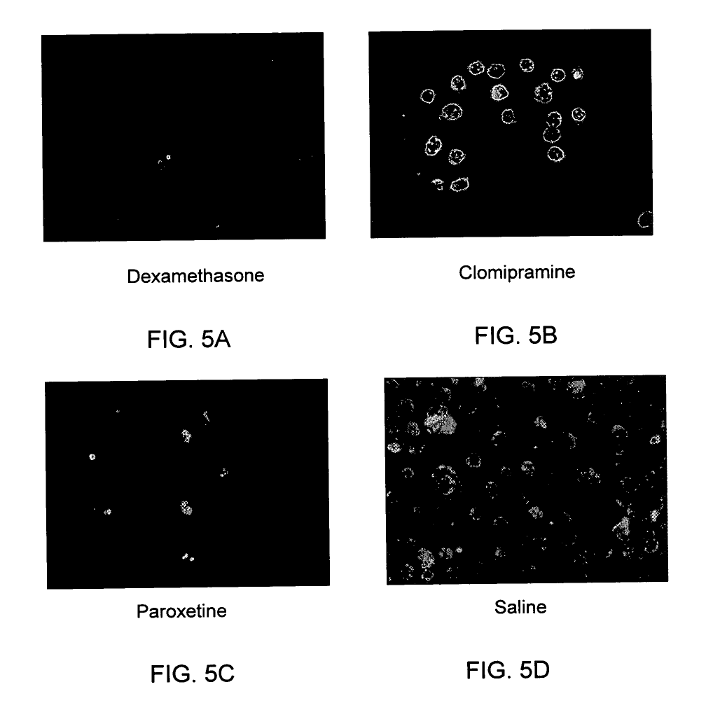

In a different assay, the apoptotic effect of clomipramine and paroxetine (20 μM) was examined with dexamethasone used as positive control for anti-inflammatory response. In particular, HaCat cells (10,000/well) were exposed to the different drugs (20 μM) or to saline (control). Twenty four hours post exposure, the cells were co-stained with the DNA binding dyes, Hoechst 33342 (20 μg/ml) propidium iodide (PI) (10 μg/ml), for 5 min at room temperature. The cells were then examined using a fluorescence microscope with ultraviolet excitation at 340-380 nm. Typically, intact cells are detected by a blue Hoechst fluorescence while fragmented nuclear is detected by a red fluorescence, indicating cells undergoing apoptosis [Harel et al., Sensitivity of HaCat keratinocytes to diabetogenic toxins, Biochem. Pharmacol., 63:171-178, 2002]. The results presented in FIG. 5 show that clomipramine and paroxetine which are antidepressants but not dexamethasone which is a glucocorticosteroid induced a dramatic increase in red fluorescence of nuclei, typical morphological changes indicating apoptosis, with paroxetine treated cells being almost totally disintegrated (FIG. 5).

An additional marker for apoptosis is the activation of caspase-3 (a key mediator implicated in apoptosis in mammalian cells belong to the asparate-specific cysteinyl proteases or caspases).

In the following assay caspase-3 activation was measured by an enzymatic fluorimetric method, using fluorigenic substrate (Ac-DEVD-AMC) (Biomol, PA, USA) which produces a blue fluorescence detected at a 360 nm wavelength. The fluorigenic moiety AMC is cleaved from the substrate in the presence of caspase-3 (and caspase-3-like enzymes), which results in appearance of a yellow-green fluorescence monitored by a fluorimeter at 460 nm. The amount of the yellow-green fluorescence is proportional to the activity of the caspase-3 in the cell extract sample.

The effect of paroxetine and sertraline on caspase-3 activation was tested in human HaCaT cells. Specifically, 1×10⁶ cells were exposed to paroxetine (10-20 μM) or sertraline (10-15 μM) for 4 hours, followed by extraction with Triton X-100 to obtain cell lysates. Measurements were determined, after the 4 hr exposure, every 3 min during 45 min, after which a specific caspase 3 inhibitor DEVD-AMC-CHO (Alexis corp. Lausen Switzerland) was added to half of the samples and measurements continued for another 45 min as previously described (Garcia-Calvo M, Peterson E P, Leiting B, Ruel R, Nicholson D W, Thornberry N A: Inhibition of human caspases by peptide-based and macromolecular inhibitors. J Biol Chem, 27(49):32608-13, 1998).

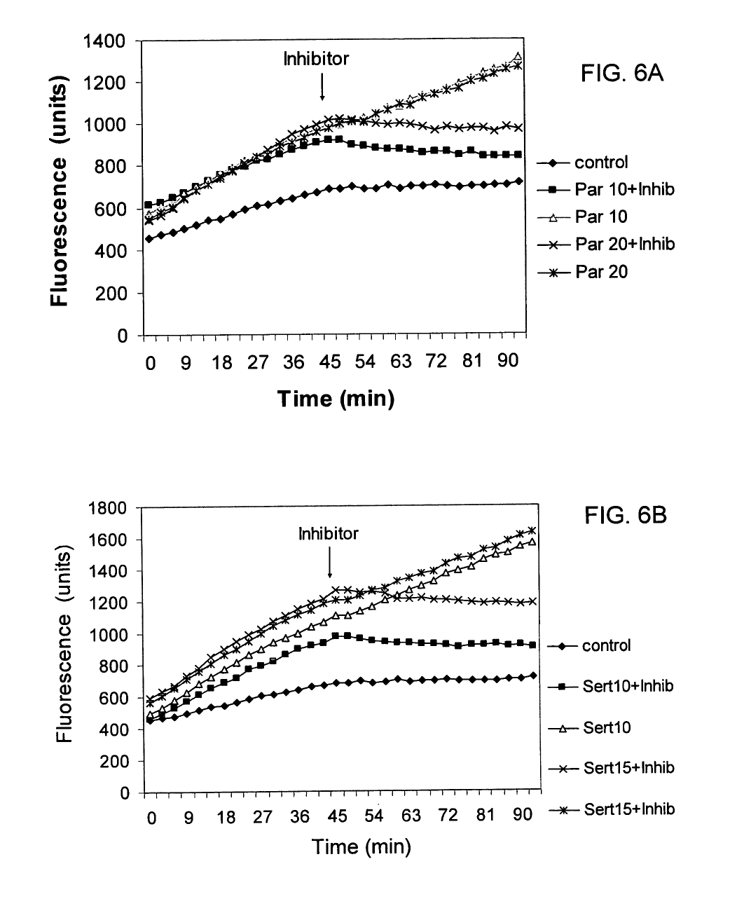

The results presented in FIGS. 6A and 6B show that paroxetine and sertraline, respectively, induce a significant increase in caspase-3 activity (which was inhibited by caspase-3 specific inhibitor). These results confirm the preceding finding showing that psychotropic drugs effectively induce apoptosis.

### Example 1C

### Effect of Cyclic Psychotropic Agents on Cell Viability in Wild Type and MDR B16 Melanoma Cells

1C(i) Tricyclic and Antipsychotic

The effect of clozapine, a tricyclic neuroleptic and antipsychotic drug, on cell viability of wild type and MDR B16 melanoma cells was examined. In particular, clozapine was applied for 24 hr to either wild type B16 melanoma cells (20,000/well) at different concentrations (10, 20, 30, 40, 50, 75 and 100 μM) or to MDR B16 melanoma cells (20,000 cells/well).

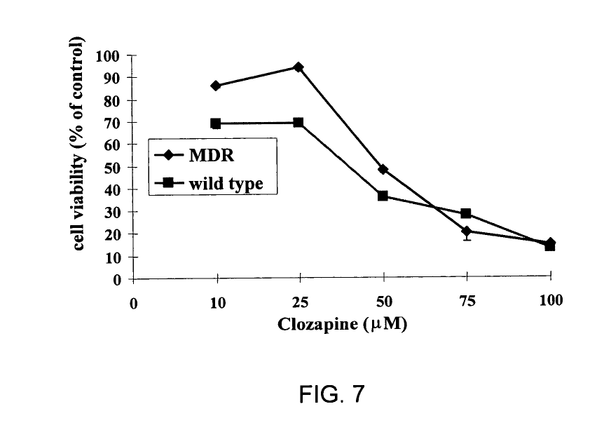

FIG. 7 summarizing the results, shows that in the presence of clozapine, the viability of the tested cell lines was reduced in a dose dependent manner.

1C(ii) Cyclic Antidepressants

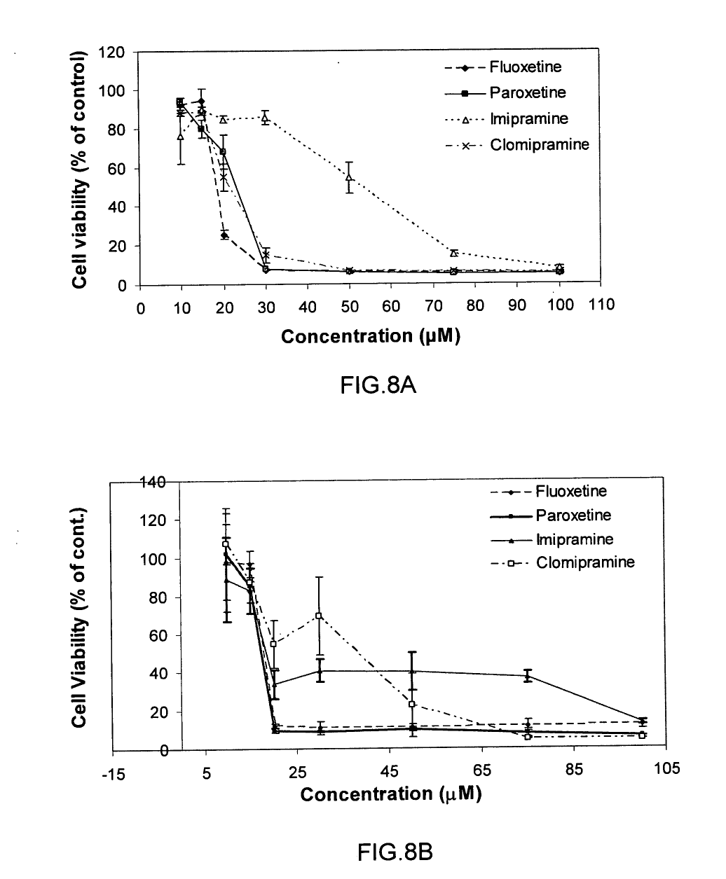

Clomipramine and imipramine (tricyclic antidepressant); paroxetine (bicyclic antidepressant); and fluoxetine (monocyclic antidepressant) were applied to wild type B16 melanoma cells (20,000/well) for 24 hr at concentrations of 10, 15, 20, 30, 50, 75 and 100 μM. FIG. 8A shows that these drugs are capable of reducing cell viability, with fluoxetine being the most effective drug.

In further assay, the effect of clomipramine, imipramine, fluoxetine and paroxetine on MDR B16 melanoma cells was tested. Specifically, the different drugs were applied for 24 hr to the cells (20,000/well) at concentrations of 10, 20, 30, 50, 75 and 100 μM. The results, presented in FIG. 8B, show a high sensitivity of the MDR cells to the cyclic antidepressant drugs, with IC50 levels of between 15-20 μM.

### Example 1D

### Sensitization of MDR B16 Melanoma Cellsto Chemotoxic Agents by Cyclic Psychotropic Drugs

The effect of clomipramine (a tricyclic antidepressant) on the toxicity of doxorubicin in wild type and MDR B16 melanoma cells was evaluated.

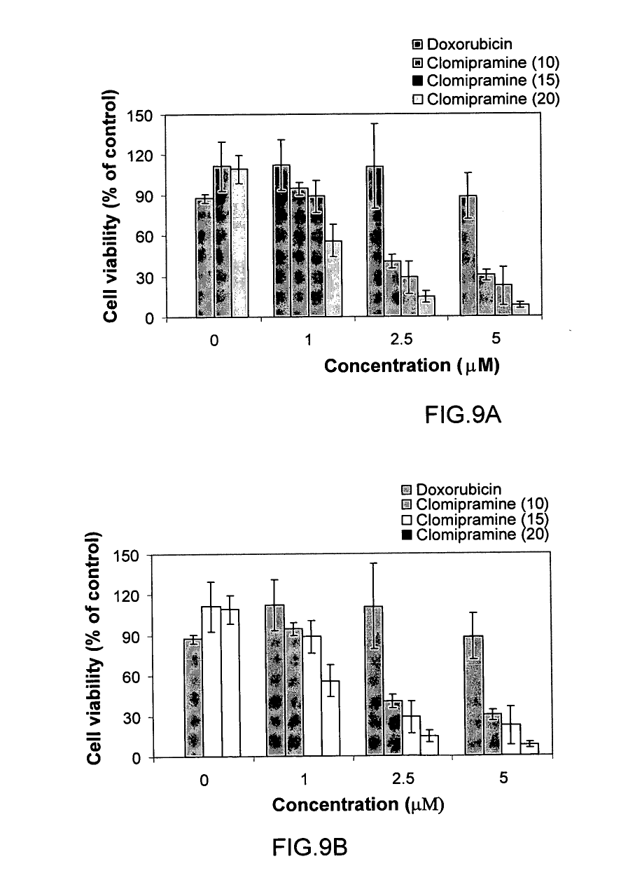

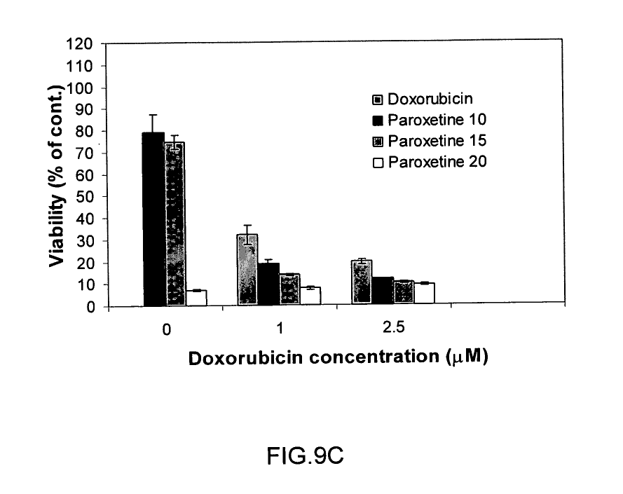

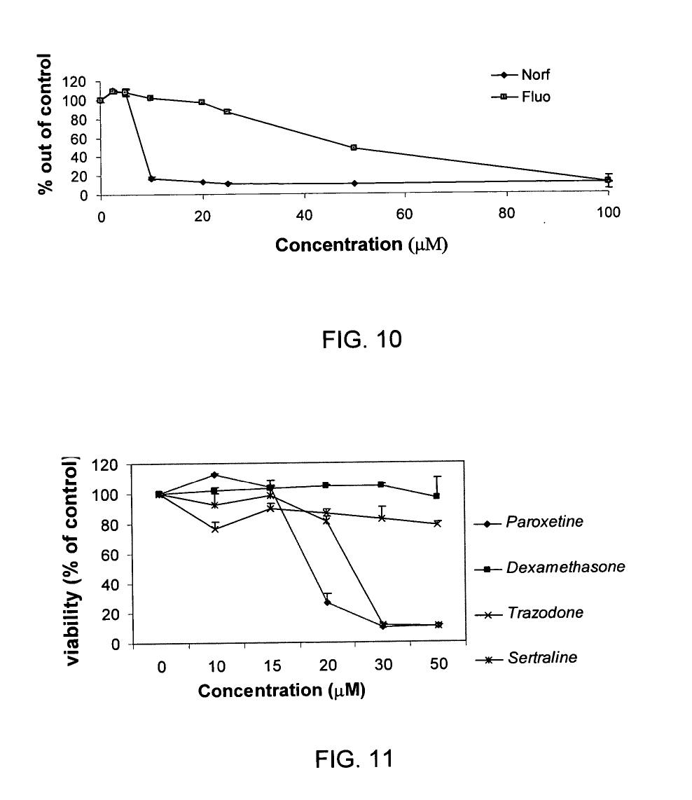

In a first assay, clomipramine (10, 15, and 20 μM) was applied to wild type B16 melanoma cells (20,000/well) for 24 hr either alone or in combination with doxorubicin (at concentrations of 1, 2.5 and 5 μM). FIG. 9A shows that the combination of clomipramine with the toxic agent, doxorubicin, resulted a further reduction in cell viability, to less than 15% from control, as compared to clomipramine alone (see FIG. 8A).

When clomipramine was applied to MDR B16 melanoma in combination with doxorubicin as described above, cell viability was also significantly reduced, in a dose dependant manner. These results, presented in FIG. 9B, demonstrate the potentiation of doxorubicin by the antidepressant.

A similar effect was observed with paroxetine when applied to wild type B16 melanoma cells, the results of which are presented in FIG. 9C.

These results teach that psychotropic drugs, and particularly cyclic antidepressants can sensitize MDR cells to subsequent administration of chemotoxic agents.

In yet a further assay, a the effect on viability of HaCat cells of the mono cyclic antidepressants fluoxetine as compared to its active metabolite norfluoxetine was examined. Norfluoxetine preserves the serotonin reuptake inhibition and has extremely long half life [Sanchez C, Hyttel J Comparison of the effect of antidepressants and their metabolites on reuptake of biogenic amines and on receptor binding. Cell. Mol. Neurobiol, 19:467-89 (1999)].

The results show structure activity relationship between the two agents, with five times increased antiproliferative activity of norfluoxetine as compared to fluoxetine, The results indicate that the anti-proliferative effect is preserved and can be augmented by small chemical changes of the active cyclic psychotropics (for example demethyl metabolites like norfluoxetine, demethylsertraline, demethylparoxetine and demethylsertraline) may change dramatically the anti-proliferative activity of the cyclic psychotropic.

### Example 2

### In Vivo Studies

### Example 2A

### Topical Formulations Comprising a Psychotropic Drug or its Corresponding Salt as Active Ingredient

The following are non-limiting examples of topical formulations which may be applied to a subject in need according to the method of the invention. The active ingredient or its salt referred to in the following formulations include a cyclic psychotropic drug or its pharmaceutically acceptable salt, hydrate or enantiomer.

2A(i) - Cream

| Constituent | Percentage |
|---|---|
| Active ingredient or its salt | 0.01-1.0% |
| Propylene glycol | 26.0% |
| Stearyl alcohol | 8.0% |
| Isopropyl myristate | 6.0% |
| Cetyl alcohol | 3.0% |
| Polysorbate 60 | 2.0% |
| Sorbian monostearate | 1.0% |
| Ascorbyl palmitate | 0.02-1.0% |
| P. water | Q.S. to 100% |

2A(ii) - Cream

| Constituent | percentage |
|---|---|
| Active ingredient or its salt | 0.01-5.0% |
| Whitle pet. | 21.5% |
| Stearyl alcohol | 15.0% |
| Popylene glycol | 11.5% |
| Polysorbate 60 | 5.0% |
| Antioxidant | 0.01-2.0% |
| Methyl paraben | 0.025% |
| Propyl paraben | 0.015% |
| P. water | Q.S. to 100% |

2A(iii) - Ointment

| Constituent | Percentage |
|---|---|
| Active ingredient or its salt | 0.01-1.0% |
| Lanoline alcohol | 3.0% |
| Stearyl alcohol | 6.0% |
| White wax | 5.0% |
| Propylene glycol | 10.0% |
| White petrolatum | Q.S. to 100% |

2A(iv) - Hydrophilic Ointment

| Constituent | Percentage |
|---|---|
| Active ingredient or its salt | 0.05-5.0% |
| Methyl paraben | 0.025% |
| Propyl paraben | 0.015% |
| Antioxidant | 0.01-2.0% |
| Sodium Lauryl sulfate | 1.0% |
| Propylene glycol | 12.0% |
| Stearyl alcohol | 25.0% |
| White petrolatum | 25.0% |
| P water | Q.S. to 100% |

2A(v) - Gel

| Constituent | Percentage |
|---|---|
| Active ingredient or its salt | 0.01-5.0% |
| Carbopol 934P | 2.0% |
| Triethanlamine | 1.65% |
| Methyl paraben | 0.2% |
| Antioxidant | 0.01-2.0% |
| P water | Q.S. to 100% |

2A(vi) - Foam

| Constituent | Percentage |
|---|---|
| Active ingredient or its salt | 0.01-5.0% |
| Antioxidant | 0.01-2.0% |
| Stearic acid | 5.9% |
| Triethanolamine | 3.1% |
| Lanolin | 1.0% |
| Glycerin | 2.0% |
| Lauric deithanolamide | 2.0% |
| P water | Q.S. to 100% |

92 parts packed with 8 parts of Butan 40

2A(vii) - Foam

| Constituent | Percentage |
|---|---|
| Active ingredient or its salt | 0.01-2.0% |
| Antioxidant | 0.01-1.0% |
| Arlacel-186 | 0.15% |
| Vrij 35 | 1.0% |
| Isopropanol | 6.54% |
| Cetiol | 9.0% |
| Transcutol | 30.0% |
| Triethanolamine to adjust | |
| pH | |
| P water | Q.S. to 100% |
| 95 parts packed with 5 parts | OK |
| propellant | |

2A(viii) - Solution

| Constituent | Percentage |
|---|---|
| Active ingredient or its salt | 0.05-5% |
| PEG 400 | 10.5% |
| Isopropanol | 31.5% |
| Propyl glycol | Q.S. to 100% |

2A(ix) - solution

| Constituent | Percentage |
|---|---|
| Active ingredient or its salt | 0.05-5% |
| Hexylene glycol | 12.0% |
| Glycerin | 38.0% |
| Isopropanol | Q.S. to 100% |

2A(x) - Lotion

| Constituent | Percentage |
|---|---|
| Active ingredient or its salt | 0.01-5% |
| Isopropanol | 40.0% |
| Propyl glycol | 30.0% |
| Hydroxypropyl cellulose | 1.5% |
| Buffer solution | Q.S. |
| Antioxidant | 0.01%-2.0% |
| P. Water | Q.S. to 100% |

### Example 2B

### Topical Treatment of Psoriatic Subjects

Two subjects suffering from psoriasis with no discernible psychiatric symptoms were treated with a cream containing thioridazine. The thioridazine cream was prepared by dissolving thioridazine (3 mg) in distilled water (1.5 ml), adding the solution to a standard (aqueosum) eucerinum preparation (30 g) and mixed thoroughly until a homogenous cream was obtained.

Two other subjects were treated with paroxetine cream. The cream was prepared by dissolving paroxetine (15-30mg) in distilled water (1.5-2.0 ml) and then mixing the solution with a standard (aqueosum) eucerinum preparation (30 g) until a homogenous cream at concentration of 0.05-0.1% was obtained.

Subject 1

An 18 years old female subject suffered since the age of 4 from localized psoriasis with scaling and erythema mainly in elbows and knees (however, otherwise healthy and with no psychiatric disturbances or symptoms). The subject responded poorly to topical steroids. After several months without treatment, the psoriatic areas of the subject's skin were applied twice a day with thioridazine cream. A marked reducing in the skin's scaling and erythema were noticed even a few days after treatment, the improvement in the skin's condition persisted for one year (using 0.01% in the first year and 0.02% during the next two years). In addition, treatment was effective in elimination lesions in the knees and reducing the size of the lesions in the elbows.

Upon cessation of the treatment (for 14 days) a marked exacerbation of the psoriatic lesions was observed which vanished after reestablishment of the treatment.

Subject 2

A 60 years old healthy male subject suffering from local psoriasis on the back and palms of his hands (however, otherwise healthy and with no psychiatric disturbances or symptoms) was treated twice daily with a thioridazine cream as described above (0.02%). After four months of treatment a decrease in the scales and erythema was observed. Cessation of treatment resulted in recurrence of the psoriatic symptoms.

Subject 3

A 71 year old woman suffering from severe plaque psoriasis on legs and hands which necessitated repeated hospitalizations and which responded poorly to topical steroids and vitamin D analogues. Therapy of this subject initiated by applying paroxetine cream (0.05%) twice daily for 10 months only the psoriatic areas. During the treatment period general amelioration was observed with no scaling, and decrease in erythema and pruritus.

Subject 4

A 54 year old female, suffering from severe spread plaque psoriasis on legs, elbows and palms, and which was resistant to current therapy (Topical steroids and Vit D analogs). Treatment including application of paroxetine cream (0.05%) twice daily for a period of 4 months. Amelioration of general skin appearance, with inhibition of scaling and erythema particularly in the palms was reported. In the following 8 months the subject was treated with paroxetine cream (0.07%) which also exhibited general improvement of symptoms and restriction of affected areas (legs and elbow), and disappearance of some lesions. Treatment of the subject with a placebo (vehicle cream only) was reported to be inefficient. No side effects were reported.

### Example 3A

### Con-A-induced Human Lymphocyte Proliferation

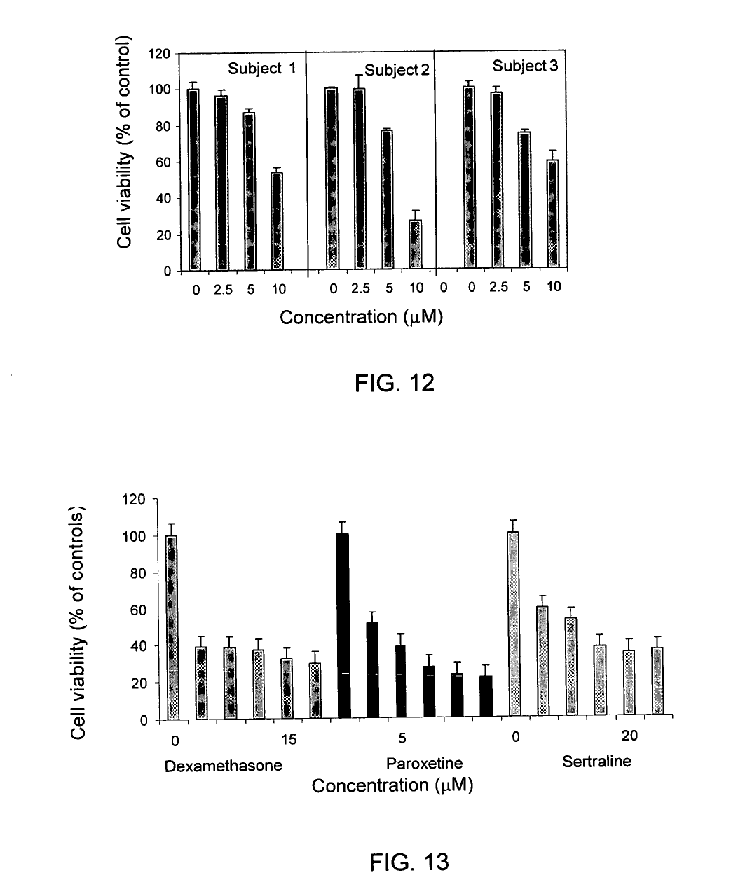

Human lymphocytes were isolated from whole blood of 3 healthy volunteers. Lymphocyte proliferation was induced by exposure 1×10⁶ cells/well to phytohemagglutinin (PHA) (2.5 μg/ml). Cell viability was assessed 48 hr post exposure to paroxetine (2.5-10.0 μM) using Alamar blue reagent (Wildflower, Santa Fe, N.M., USA) staining Nociari M M, Shalev A, Benias P , et al. A novel one-step highly sensitive fluorimetric assay to evaluate cell-mediated cytotoxicity. J. Immunol. Methods 213(2): 157-167, 1998. The results presented in FIG. 12 clearly show that paroxetine was able to inhibit human lymphocyte-induced proliferation in a dose dependent manner with IC50 of 5-10 μM.

### Example 3B

### Concavalin A (Con-A)-induced Mouse Splenocyte Proliferation

The effect of several psychotropic drugs on the proliferation of Con-A-induced mouse splenocyte proliferation was examined. The specific drugs evaluated included, without being limited thereto, the bicyclic antidepressants paroxetine and sertraline.

In particular, mouse splenocytes were isolated from C57BL healthy female mice. Cells (10,000/well) were then exposed to Con-A (5H) and treated with a vehicle 2.5-10 μm, dexamethasone or with a drug (2.5-10 μM).

Cell viability was assessed 48 hr later using Alamar blue staining. The results presented in FIG. 13 (each point is the graph represents the mean of 4 determinations) show that the bicyclic antidepressants paroxetine and sertraline induce a dose-dependent decrease in mitogen-induced splenocyte mouse proliferation in a similar pattern to the corticosteroid dexamethasone. The IC50 determined for the antidepressant drugs was in the range of 2.5-5.5 μM.

### Example 3C

### (Con-A)-induced Mouse Splenocyte Proliferation and Secretion of IL-2 and INF-γ

The effect on Con-A-induced splenocyte proliferation (determined by Alamar blue method) and on splenocyte-induced secretion of IL-2 and INF-γ of paroxetine (bicyclic antidepressant) and clomipramine (tricyclic antidepressant) in comparison with dexamethasone was evaluated. In particular, cells 1×10⁶ cells/well were exposed for 48 hours to each drug (2.5-10 μM).

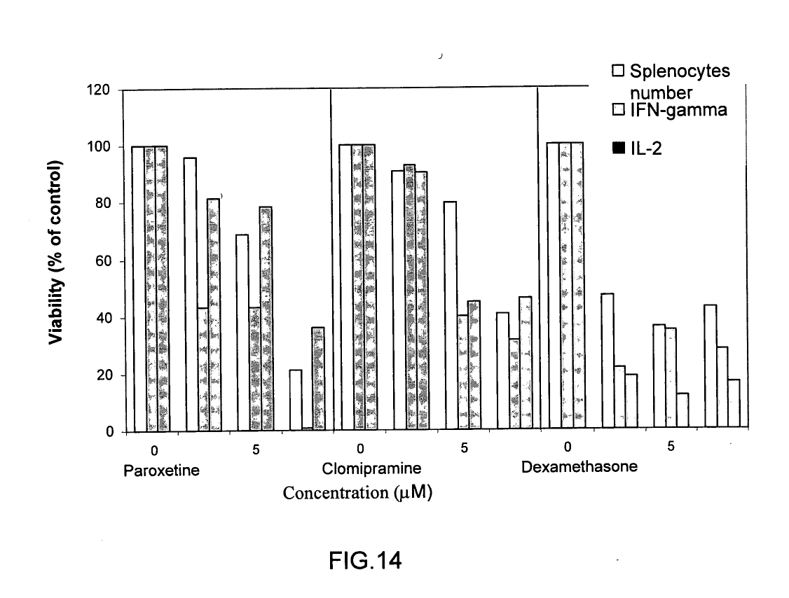

The results are presented in FIG. 14 which show that that paroxetine and clomipramine caused a dose dependent decrease in splenocyte proliferation leading to a decrease in the lymphocyte-induced cytokine secretion in a similar pattern to that observed by dexamethasone

### Example 3D

### (Con-A)-induced TNF-α Secretion

The effect on ConA-induced TNF-α secretion of paroxetine, sertraline, thioridazine, in comparison with dexamethasone (all at concentrations of 2.5-20 μM) was also determined. Cells treated with Con-A alone served as the control (“Con-A”).

In particular, mouse splenocyte cells 1×10 (cells/well) were exposed, for 48 hours, to the different drugs and ConA and at the end of the experiment plates were centrifuged and supernatant collected for cytokine determination and TNF-α levels were determined in soups by ELISA. (R&D #DY410 Minnapolis USA)

Values are expressed in percent out of control ((Vehicle-ConA treated cells), data represent mean+SE of three experiments. The sensitivity of the assay was 32 pg/ml, basal levels were below the assay sensitivity, and the ConA induced TNF-α stimulation levels were 1067 pg/ml.

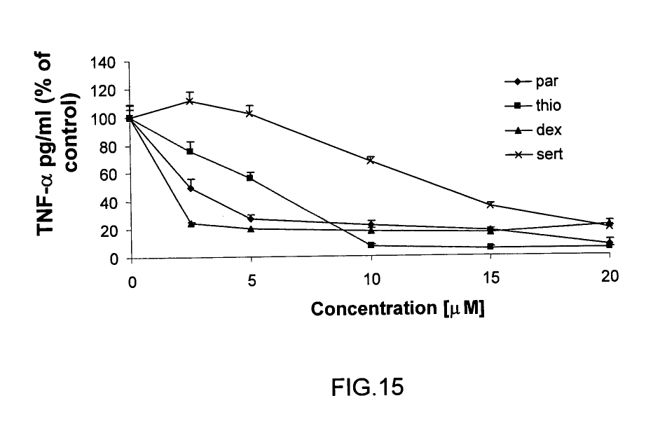

FIG. 15 presents the results for each drug (mean+SE of three experiments, presented as percent out of the control). As exhibited in FIG. 15, the drugs caused a dose-dependent decrease in TNF-α levels with paroxetine showing almost a similar pattern as compared to dexamethasone.

Results show that all agents caused a dose dependent decrease in TNF-α levels with paroxetine showing almost a similar pattern as compared to dexamethasone. In particular, IC50 levels for the different drugs were 2.5, 7.3 and 14.3 μM, respectively, for paroxetine, thioridazine and sertraline compared to the IC50 of dexamethasone, being 0.6 μM.

These results may suggest that psychotropic drugs affect the immune system via a glucocorticoid-like mechanism.

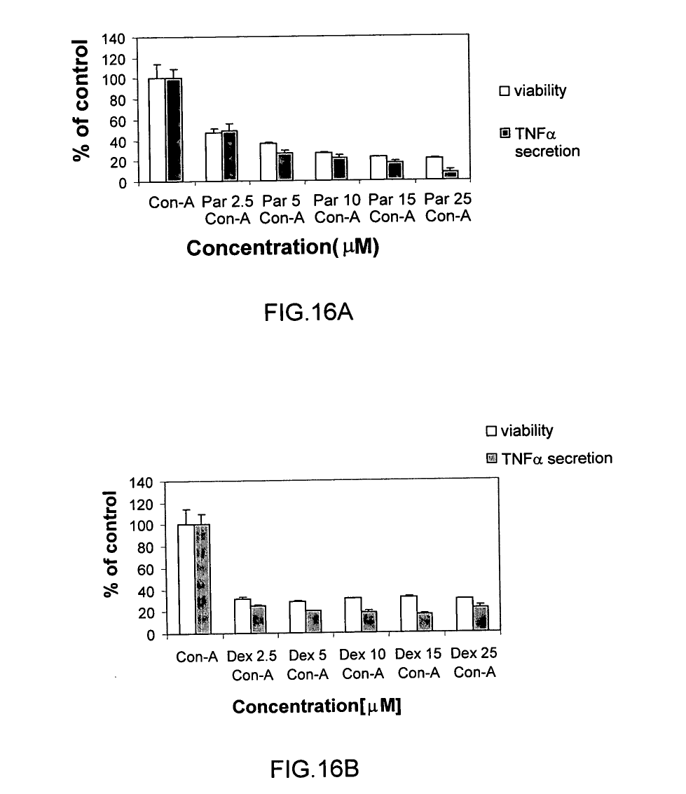

In yet a further assay, the effect on Con-A-induced splenocyte proliferation (Almar Blue method) and on splenocyte-induced secretion of TNF-α, of paroxetine, with comparison to dexamethasone (each at concentrations of between 2.5-25 μM) was evaluated. As described above, cells were exposed to each drug for 48 hours. FIG. 16A and FIG. 16B show the effect of paroxetine and dexamethazone, respectively, on cell viability (proliferation) and TNF-α secretion.

As shown in FIG. 16A, paroxetine inhibited cell proliferation and TNF-α secretion in a dose dependent manner and that there is a high correlation between the effect on proliferation and TNF-α secretion. The effect of paroxetine also correlated with the effect of dexamethasone (FIG. 16B), suggesting that it affects cells via a similar pathway.

1. A topical pharmaceutical composition, comprising a topically acceptable carrier, and at least one active ingredient, a cyclic psychotropic agent, said cyclic psychotropic agent being other than doxepine and tomoxetine.

2. A composition according to claim 1, wherein the cyclic psychotropic agent is an anti-depressant.

3. A composition according to claim 2, wherein active anti-depressant is selected from: selective serotonin re-uptake inhibitors (SSRIs); selective noradrenaline re-uptake inhibitors (NRISs), serotonin and noradrenergic re-uptake inhibitors (SNRIs); cyclic anti-depressants, and atypical anti-depressant.

4. A composition according to claim 3, wherein:
(a) the SSRIs are selected from: fluoxetine, paroxetine and sertraline;
(b) the NRISs being: reboxetine
(c) the SNRI is selected from: venlafaxine, duloxetine and milnacipran;
(d) the cyclic anti-depressant selected from:
(d1) tricyclic anti-depressant selected from: imipramine, clomipraminne, amitriptyline and doxepinee;
(d2) bicyclic anti-depressants selected from: paroxetine, sertraline;
(d3) monocyclic anti-depressants selected from: : phenylpropylamine derivatives ;The composition according to claim 4, wherein the phenylpropylamine derivates are phenoxy-3-propylamine derivatives.
(d4) atypical antidepressants selected from: mianserin, bupropion, mirtazaoin, trazodone.

5. The composition according to claim 4 wherein the phenylpropylamine derivates are phenoxy-3-propylamine derivatives.

6. The composition according to claim 5, wherein the phenoxy-3 proplylamine derivatives are selected from: nisoxetine, fluoxetine, norfluoxetine, reboxetine, atomoxetine and venlafaxine.

7. The composition according to claim 1, wherein the cyclic psychotropic agent is an anti-psychotic drug.

8. The composition according to claim 7, wherein the anti-psychotic drug is selected from tricyclic anti-psychotic drug and atypical antipsychotic drug.

9. The composition according to claim 8, wherein the tricyclic anti-psychotic drug is phenothiazine.

10. The composition according to claim 9, wherein the phenothiazine is selected from: thioridazine, perphenazine, trifluoperazine and fluphenazine.

11. The composition according to claim 8, wherein the tricyclic antipsychotic drug is thioxanthenes.

12. The composition according to claim 11, wherein the thioxanthenes are selected from flupenthixol, thiothixene, chlorprothixene and zuclopentihixol.

13. The composition according to claim 8, wherein the atypical anti-psychotic drug is selected from clozapine, quetiapine, ziprazidone, olanzapine and risperidone.

14. A composition according to claim 1 in a formulation selected from: ointment, cream, gel, solution, suspension, lotion, shampoo, foam, lyposomic formulation, paste, emulsion, salve, suppositories, vaginal tablets, ocular salves or drops, otic drops, nasal spray and nasal drops.

15. A composition according to claim 14 in a formulation selected from: cream, ointment, gel, foam, solution, lotion.

16. A method for the treatment of a dermatological disease, disorder, or pathology the method comprising, topically administering to a subject in need of dermatological treatment, a therapeutically effective amount of a psychotropic cyclic agent, said cyclic psychotropic agent being other than atomoxetine and doxepine.

17. A method for the treatment of hyper-proliferative dermatological diseases, disorders or pathological conditions, comprising topically administering to a subject, in need of such treatment, a therapeutically effective amount of a cyclic psychotropic agent,
wherein, where the hyper-proliferative skin disorder is psoriasis, the cyclic psychotropic agent is not atomoxetmne.

18. A method according to claim 17, wherein said hyperproliferative skin disease or disorder is selected from: psoriasis, scerloderma, epidermal hyperplasia, hyperkeratosis, acanthosis, papilloma, actinic keratoses, and skin cancer.

19. The method according to claim 18, wherein said skin cancer is selected from basal cell carcinoma, melanoma, squamous cell carcinoma, cutaneous T-cell lymphoma and Kaposi's sarcoma.

20. The method according to claim 17, wherein the cyclic psychotropic agent is an anti-depressant.

21. The method according to claim 20, wherein the anti-depressant is selected from: selective serotonin re-uptake inhibitor (SSRI); selective noradrenaline re-uptake inhibitor (NRIS), serotonin and noradrenergic re-uptake inhibitor (SNRI); cyclic anti-depressants, and atypical anti-depressant.

22. The method according to claim 21, wherein:
(a) the SSRI is selected from fluoxetine, paroxetine and sertraline;
(b) the NRIS is selected from: atomoxetine and reboxetine;
(c) the SNRI is selected from venlafaxine, duloxetine and milnacipran;
(d) the cyclic anti-depressant is selected from:
(d1) tricyclic anti-depressant selected from: imipramine, clomipraminne, amitriptyline and doxepinee;
(d2) bicyclic anti-depressants selected from: paroxetine, sertraline and citalopram;
(d3) monocyclic anti-depressants selected from: phenylproply derivatives, fluoxetine and norfluoxetine
(d4) atypical anti-depressants selected from:: mianserin, bupropion, mirtazaoin and trazodone.

23. The method according to claim 22, wherein the phenylpropylamine derivates are phenoxy-3-propylamine derivatives.

24. The method according to claim 23, wherein the phenoxy-3 proplylamine derivatives are selected from: atomoxetine, nisoxetine, fluoxetine, norfluoxetine, reboxetine and venlafaxine.

25. The method according to claim 17, wherein the cyclic psychotropic agent is an anti-psychotic drug.

26. The method according to claim 25, wherein the anti-psychotic drug is selected from tricyclic anti-psychotic drug and atypical antipsychotic drug.

27. The method according to claim 26, wherein the tricyclic anti-psychotic drug is phenothiazine.

28. The method according to claim 27, wherein the phenothiazine is selected from: thioridazine, perphenazine, trifluoperazine and fluphenazine.

29. The method according to claim 26, wherein the tricyclic antipsychotic drug is thioxanthenes.

30. The method according to claim 29, wherein the thioxanthenes are selected from flupenthixol, thiothixene, chlorprothixene and zuclopentihixol.

31. The method according to claim 26, wherein the atypical anti-psychotic drug is selected from clozapine, quetiapine, ziprazidone, olanzapine and risperidone.

32. A method for the treatment of an inflammatory dermatological disease, disorder or pathological condition comprising topically administering to a subject, in need of such treatment, a therapeutically effective amount of a cyclic psychotropic agent,
wherein, where the inflammatory skin disorder, disease or pathological condition is manifested by pruritus, the cyclic psychotropic agent is not doxepine.

33. A method according to claim 32 wherein, where the inflammatory skin disorder, disease or pathological condition is manifested by pruritus, or the skin disorder is atopic dermatitis the cyclic psychotropic agent is not doxepine.

34. A method according to claim 33, wherein the inflammatory disease, disorder or pathological condition is an autoimmune disease.

35. A method according to claim 34, wherein said autoimmune skin disorder is selected from: vitiligo, scerloderma, alopecia areata, psoriatic arthritis, lichen planus, lichen sclerosus, discoid lupus, lupus erythematosus, leg ulceration in rheumatoid arthritis, atopic dermatitis, cicatrical pemphigoid and pyoderma gangrenosum.

36. A method according to claim 34, wherein the inflammatory disease is a non-autoimmune disease.

37. A method according to claim 36, wherein the inflammatory disease is selected from: rosacea, pruritus, seborrheic dermatitis and contact dermatitis .

38. A method according to claim 33, wherein the cyclic psychotropic agent is an anti-depressant.

39. A method according to claim 38, wherein the active anti-depressant is selected from: selective serotonin re-uptake inhibitor (SSRI); selective noradrenaline re-uptake inhibitor (NRIS), serotonin and noradrenergic re-uptake inhibitor (SNRI); cyclic anti-depressants, and a typical anti-depressant.

40. A method according to claim 39, wherein:
(a) the SSRI is selected from: fluoxetine, paroxetine, sertraline;
(b) the NRIS is selected from: atomoxetine and reboxetine;
(c) the SNRI is selected from: venlafaxine, duloxetine and milnacipran;
(d) the cyclic anti-depressant selected from
(d1) tricyclic anti-depressant selected from imipramine, clomipraminne, amitriptyline and doxepine;
(d2) bicyclic anti-depressants selected from paroxetine, sertraline and citalopram;
(d3) monocyclic anti-depressants are phenylpropylamine derivatives fluoxetine and norfluoxetine.
(d4) atypical anti-depressants selected from mianserin, bupropion, mirtazaoin and trazodone.

41. A method according to claim 40, wherein the phenylpropylamine derivates are phenoxy-3-propylamine derivatives.

42. A method according to claim 41, wherein the phenoxy-3 proplylamine derivatives are selected from: atomoxetine, nisoxetine, fluoxetine, norfluoxetine, reboxetine and venlafaxine.

43. A method according to claim 33, wherein the cyclic psychotropic agent is an anti-psychotic drug.

44. A method according to claim 43, wherein the anti-psychotic drug is selected from tricyclic anti-psychotic drug and atypical antipsychotic drug.

45. A method according to claim 44, wherein the tricyclic anti-psychotic drug is phenothiazine.

46. A method according to claim 45, wherein the phenothiazine is selected from: thioridazine, perphenazine, trifluoperazine and fluphenazine.

47. A method according to claim 43, wherein the tricyclic antipsychotic drug is thioxanthenes.

48. A method according to claim 47, wherein the thioxanthenes are selected from flupenthixol, thiothixene, chlorprothixene and zuclopentihixol

49. A method according to claim 43, wherein the atypical anti-psychotic drug is selected from clozapine, quetiapine, ziprazidone, olanzapine and risperidone.

50. A method for sensitizing skin cancer cells to chemotoxic drugs, the method comprising topically administering to a subject, in need of chemotoxic therapy a therapeutically effective amount of a cyclic psychotropic agent, with the proviso that the cyclic psychotropic agent is not fluoxetine.

51. A method according to claim 50 wherein the skin cancer is multi-drug resistant skin cancer.

52. A method according to claim 50 wherein the cyclic psychotropic drug is topically administered simultaneously with the administration of the chemotoxic drug.

53. A method according to claim 50 wherein the cyclic psychotropic drug is topically administered prior to the administration of the chemotoxic drug.

54. A method according to claim 50 wherein the chemotoxic drug is administered systemically.

55. A method according to claim 50 wherein the cyclic psychotropic agent is a cyclic anti-psychotic drug.

56. A method according to claim 55 wherein the anti-psychotic drug is a tricyclic antipsychotic drug.

57. A method according to claim 56 wherein the trycyclic antipsychotic drug is phenothiazine.

58. A method according to claim 57, wherein the phenothiazine is selected from: thioridazine, perphenazine, trifluoperazine and fluphenazine

59. A method for identifying and screening for , an active agent for the treatment of a dermatological/mucosal disease, disorder or pathological condition by topical or mucosal application, the method comprising:
(a) providing one cyclic psychotropic drug as a candidate active agent;
(b) applying the cyclic psychotropic drug to a biological model system for said dermatological/mucosal disease, disorder or pathological condition;
(c) monitoring the change in at least one physiological parameter, said change being indicative of a beneficial therapeutic effect in said biological model system;
wherein a significant change in said at least one physiological parameter as compared to control indicates that the candidate cyclic psychotropic agent is active for the treatment of said dermatological disease, disorder or pathological condition.

60. A method according to claim 59 wherein the cyclic psychotropic drug is an anti psychotic drug or an antidepressant.
